(19) United States
(12) Patent Application Publication
(10) Pub. No.: US 2014/0005116 A1
(43) Pub. Date: Jan. 2, 2014

(54) KELOID TREATMENT

(71) Applicants: Adebola O.E. Obayan, Saskatchewan (CA); Sanjeev Singh, Saskatchewan (CA); Brenda Collen Joyce Yuzdepski, Saskatchewan (CA)

(72) Inventors: Adebola O.E. Obayan, Saskatchewan (CA); Sanjeev Singh, Saskatchewan (CA); Brenda Collen Joyce Yuzdepski, Saskatchewan (CA)

(73) Assignee: ADE THERAPEUTICS INC., Saskatchewan (CA)

(21) Appl. No.: 13/807,378

(22) PCT Filed: Jun. 28, 2011
(86) PCT No.: PCT/CA11/50396

§ 371 Date: Sep. 11, 2013

Related U.S. Application Data
(60) Provisional application No. 61/359,053, filed on Jun. 28, 2010.

(51) Int. Cl. A61K 38/05

(52) U.S. Cl. CPC A61K 38/05
USPC 514/18.6

(57) ABSTRACT

Due to a long-standing interest in scar reduction, alanyl-glutamine was tested for ability to treat keloid scars, despite the difficulties associated with delivering compounds above a certain size threshold across the skin barrier to the dermis where new skin is formed. Surprisingly, it was found that a dermal application formulation of alanyl-glutamine was effective in reducing the severity and/or preventing the formation of keloid scars.

### PRIOR APPLICATION INFORMATION

The instant application claims the benefit of U.S. Provisional Patent Application 61/359,053, filed Jun. 28, 2010.

### BACKGROUND OF THE INVENTION

Adhesions are abnormal deposits of fibrous tissues that form within the peritoneal cavity. Abdominal adhesions are a common cause of small bowel obstruction and female infertility (Thompson and Whawell, 1995, Br J Surg 82: 3-5; Thompson, 1995, Lancet 346: 1382; Ellis, 1997, Eur J Surg Suppl 577: 5-9).

The peritoneum is composed of two mesothelial sheets that enclose predominantly adipocytes embedded in loose connective tissue, and also aggregates of mononuclear phagocytic cells. The greater omentum is the largest part of the peritoneum with the size varying from 300 gm to 2000 gm and a surface area of 300 cm² to 1500 cm². The omentum has a rich vascular supply with numerous characteristic capillary convolutions that are termed omental glomeruli due to their similarity with renal glomeruli. These capillary beds lie directly under the mesothelium (Ackermann et al., 1991, Acta Anat (Basel) 140: 146-9). Adhesions are formed as a result of fibrous repair of peritoneal injury mostly after surgery.

Factors associated with the formation of post-surgical adhesions include trauma, thermal injury, infection, ischemia and foreign bodies. Other factors associated with adhesion formation include tight suturing where tension within the sutured peritoneum produces ischemia and abrasion. Exposure to foreign bodies such as talc and powders from the gloves, lint from abdominal packs or disposable paper items may also contribute to the formation of adhesions (Menzies, 1992, Surg Annu 24: 27-45; Bridges et al., 1965, Acta Anat (Basel) 61: 203-12; Drollette and Badawy, 1992, J Reprod Med 37: 107-21). Neutropenia is associated with lower rates of adhesion and may play a role in modulating post-operative adhesion (Vural et al., 1999, Hum Reprod 14: 49-54).

Adhesion formation begins with injury inflicted on the peritoneum whether by an injurious stimuli including bacterial, chemical toxicity, ischemia, mechanical or simply drying from exposure (Williams and White, 1986, Curr Probl Surg 23: 789-865; Dijkstra et al., 2000, Scand J Gastroenterol Suppl 232: 52-9). The injury leads to an inflammatory response, which progresses to fibrin deposition and subsequent fibrinous adhesion. If the fibrinous adhesion is not degraded within the first days of the injury, reparative cells including fibroblasts are propagated into the fibrin matrix turning it into permanent fibrous adhesion. This process is completed within a week of the injury. The balance of fibrin deposition and breakdown is therefore crucial in the early phase of peritoneal repair and adhesion formation (Dijkstra et al., 2000; Vipond et al., 1990, Lancet 335: 1120-2; Whawell and Thompson, 1995, Eur J Surg 161: 315-8).

Glutamine is a conditional essential amino acid which the body is unable to synthesize in sufficient quantities under certain physiologic circumstances (Smith, 1990, J Parenter Enteral Nutr 14: 40S-44S; Lacey and Wilmore, 1990, Nutr Rev 48: 297-39) such as major surgery, shock, traumatic injury and severe sepsis. A decrease in extracellular glutamine impairs the function of macrophages and other immune cells, resulting in increased protein degradation from skeletal muscle (Newsholme, 1996, Experientia 52: 455-9). Macrophages are extremely active cells (10 times per minute based on ATP turnover and 5 times per minute based on oxygen consumption) with a high capacity to take up glutamine and ‘trap’ it as glutamate, which acts as an intracellular store for both energy formation and provision of precursors for biosynthesis. Mouse peritoneal macrophages have been shown to utilize a high amount of glutamine via the process of glutaminolysis even though they are seen as terminally differentiated cells (Newsholme et al., 1985, Biosci Rep 5: 393-400; Newsholme et al., 1985, Q J Exp Physiol 70: 473-89). These macrophages are characterized by high rate of protein secretion and membrane recycling (Werb and Cohn, 1972, J Biol Chem 247: 2439-46; Ardawi and Newsholme, 1983, Biochem J 212: 835-42). Although glutamine constitutes >50% of the unbound amino acid pool in human skeletal muscle, rapid reduction in blood and tissue glutamine has been noted following catabolic events such as major surgery (Vinnars et al., 1975, Ann Surg 182: 665-671), trauma (Askanazi et al., 1980, Ann Surg 191, 465-72), and sepsis (Roth, 1985, Z Exp Chir Transplant Kunstliche Organe 18: 150-6; Fukuzawa et al., 1995, Transplantation 59: 6-9).

Glutamine is safe, well absorbed, and has no documented side effects. Glutamine is known to enhance wound healing. Glutamine and its dipeptides have been used for parenteral and enteral supplementation components in critically ill patients. A recent study by Fukuzawa et al (1995) concluded that glutamine enhances both phagocytosis and the production of Reactive Oxygen Intermediates (ROI) by neutrophils in post operative patients. In a randomized prospective study, Morlion et al. using glutamine dipeptides in total parenteral nutrition (TPN) concluded that the supplement group had shorter hospital stay, improved immune status and nitrogen balance after abdominal surgery (Morlion et al., 1998, Ann Surg 227: 302-8).

Alanyl-glutamine and glycyl-glutamine are two dipeptides of glutamine which have been employed clinically due to their higher solubility and chemical stability over free glutamine, making them more stable sources of the constituent amino acids (Morlion et al., 1998, Ann Surg 227: 302-8; Furst et al., 1989, Metabolism 38: 67-72; Karner and Roth, 1989, Metabolism 38: 73-7; Babst et al., 1993, J Parenter Enteral Nutr 17: 566-74; Nordfjeld et al., 1983, J Clin Hosp Pharm 8: 265-74; Cardona Pera, 1998, Nutr Hosp 13: 8-20). Enteral supplementation with alanyl-glutamine but not glutamine+alanine mixture promotes intestinal adaptation as evidenced by increased peptide transport after intestinal resection (Satoh et al., 2003, J Mol Med 12: 615-20). Alanyl-glutamine also prevents intestinal damage, as demonstrated by increased peptide transport expression and an elevated plasma glutamine concentration after CPM administration (Satoh et al., 2003, J Gastroenterol Hepatol 18: 719-25). Alanyl-glutamine alone was recently used enterally in post-operative patients for the first time with reported safety (Obayan, A. O. E., Oxidative Stress: Natural History and Modulation in Surgery and Trauma Patients. (University of Saskatchewan: 2004).

### SUMMARY OF THE INVENTION

According to an aspect of the invention, there is provided a method of treating or preventing or prophylactically treating a keloid scar comprising topically administering to a site of keloid scar formation or potential keloid scar formation an effective amount of a glutamine source.

In a preferred embodiment, there is provided a method of treating or preventing or prophylactically treating a keloid scar comprising topically administering to a site of keloid scar formation or potential keloid scar formation an effective amount of L-alanyl-L-glutamine in an organogel.

In yet another aspect of the invention, there is provided a pharmaceutical composition comprising a source of glutamine arranged for topical administration.

In a preferred embodiment, the pharmaceutical composition comprises L-alanyl-L-glutamine in an organogel.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As used herein, the term ‘glutamine source’ includes L-glutamine and its physiologically acceptable salts, as well as peptides comprising L-glutamine, for example glutamine-containing dipeptides or glutamine dipeptides such as those known in the art and discussed herein. While L-glutamine may be employed in this invention, this amino acid has a relatively low water solubility (36 g/L at 20° C.) and tends to break down during sterilization and prolonged storage. Dipeptides comprising L-glutamine which are capable of being metabolized to provide L-glutamine may also be employed in this invention. Examples of such dipeptides include but are by no means limited to alanyl-dipeptide and glydipeptide. Thus, in some preferred embodiments of the invention, L-glutamine or a dipeptide comprising glutamine such as for example alanyl-dipeptide and glydipeptide are administered to the individual in need of treatment, as discussed herein. Preferably, such peptides will exhibit increased solubility in water over that of L-glutamine. Often, such peptides will also exhibit increased resistance to breakdown during sterilization and storage. Two such peptides that may be used in this invention are dipeptides comprising L-glutamine and either L-alanine or glycine. The dipeptide alanyl-glutamine (glutamine residue at the C-terminal position) has high solubility in water (568 g/L). Glycyl-glutamine (glutamine at the C-terminal position) also shows enhanced solubility in water as compared to glutamine (154 g/L). Each of the latter dipeptides is sufficiently stable during heat sterilization and prolonged storage.

As used herein, ‘organogel’ refers to a non-crystalline, non-glassy thermoreversible (thermoplastic) solid material composed of a liquid organic phase entrapped in a three-dimensionally cross-linked network. One illustrative example of such a solid material would be a capsule embedded by gelatine. The liquid can be, for example, an organic solvent, mineral oil or vegetable oil.

Pharmaceutically acceptable preparations of L-glutamine and L-glutamine containing peptides (including alanyl-glutamine) are commercially available. In addition, L-glutamine containing peptides for use in this invention may also be synthesized according to known methodology and purified and sterilized for pharmaceutical use.

As discussed in Published PCT Application WO 2007/016791, published Feb. 15, 2007 and Published PCT Application WO 2008/098364, published Aug. 21, 2008, both of which are incorporated herein by reference in their entirety, it has been discovered that intraperitoneal administration of a glutamine source will reduce post-operative adhesion formation. As discussed therein, previous therapies involving glutamine administration have employed either enteral administration (e.g. in a food supplement) or parenteral administration through the intravenous route (e.g. administration of a total parenteral nutrition formulations).

As noted above, adhesions form as a natural part of the body's healing process after surgery. As part of the process, the body deposits fibrin onto injured tissues. The fibrin acts like a glue to seal the injury and encourage deposition of cellular matrix but may also cause tissues that should be separate to adhere to one another, held together by an adhesion.

It is of note that although the adhesion study indicated that AG modulates fibroblast behaviour, prior to this study, there was no evidence that AG could be used for treating scar formation.

As will be apparent to one of skill in the art, any peritoneal injury can result in a fibrous adhesion. As discussed therein in our earlier patent application, the glutamine needed to be formulated differently for peritoneal application, specifically, thickened so as to have a greater viscosity than formulations suitable for intravenous application. Specifically, the glutamine is intended to be applied once to specifically prevent or reduce adhesion formation following insult or injury, for example, following surgery.

Keloids and keloid scars on the other hand are caused by an overgrowth of granulation tissue (collagen type III). The keloids typically form firm, rubbery lesions or shiny fibrous nodules on the site of a healed skin injury but can also expand over normal skin. The keloids can also be accompanied by physical discomfort, for example, severe itchiness and sharp pains.

Specifically, keloids are characterized by a collection of atypical fibroblasts with excessive deposition of extracellular matrix components, especially collagen, fibronectin, elastin and proteoglycans.

Furthermore, as will be appreciated by one of skill in the art, such a thickened formulation is ill-suited for transdermal application.

Fibrin is a fibrous, insoluble protein formed during normal blood clotting that is the essence of the clot. It is made from fibrinogen, a soluble plasma glycoprotein made in the liver. Collagen is a group of natural occurring proteins and the main protein of connective tissue, skin, tendons, cartilage and bone.

However, one of the inventors has a long-standing interest in scar reduction formulae and the properties of AG at reducing adhesion formation intrigued this inventor. The inventor elected to try the composition on treating keloid scars, despite knowledge of the difficulties associated with delivering compounds above a certain size threshold across the skin barrier to the dermis where new skin is formed. Surprisingly, it was found that a dermal application formulation comprising 20-25% AG (which has a molecular weight of 217.22) was effective, as discussed below.

Accordingly, in a preferred embodiment, there is provided a pharmaceutical composition for the treatment, prevention, and/or prophylactic treatment of keloid scars comprising an effective amount of AG or another suitable glyceride-containing dipeptide, for example but by no means limited to alanyl-dipeptide and glydipeptide. In some embodiments, the ‘effective amount’ of AG is an amount sufficient to reduce keloid scar formation and/or severity of the scar in an injured area compared to a scar formed as a result of a similar but untreated injury. In some embodiments, the effective amount may be for example 5-35%, 5-30%, 10-35%, 10-30%, 5-25%, 10-25% or 20-25% AG In a preferred embodiment, the pharmaceutical composition comprises an effective amount of AG and an organogel, as discussed below.

As will be appreciated by one of skill in the art, reformulation of a pharmaceutical composition to change the mode of application from interperitoneal to dermal is not a trivial matter. Specifically, not only are the additives different, in one case, the composition is applied only once (interperitoneal application) while in the other (dermally), the composition may be applied as frequently as desired by the patient.

The amino acid glutamine has been studied extensively and has been shown to be useful in treatment of serious illnesses, injury, trauma, burns, and treatment related side-effects of cancer as well as in wound healing for postoperative patients. Since it is normally produced in the liver and the lungs it is not considered an essential amino acid. When the body is stressed, it releases the hormone cortisol into the bloodstream. High levels of cortisol can lower your body's stores of glutamine which is the major fuel source of enterocytes, lymphocytes, and macrophages all necessary in proper wound healing. It is when the body is under stress that the body's requirement for glutamine may increase beyond the body's ability to synthesize it therefore requiring supplementation.

Historically, organogels have been used as a topical vehicle allows for site-specific delivery of bioactives into the deeper layers of the skin allowing for a non-invasive, convenient and relatively adverse-event free therapeutic option for a variety of conditions. As discussed above, alanyl-glutamine and glycyl-glutamine have higher solubility and chemical stability over free glutamine and so are more freely soluble in the aqueous phase of organogels. This allows higher concentrations of glutamine to be delivered across the skin barrier to the site of scar formation. Without using the correct transportation method such as an organogel, there would be no reason to expect washing a wound with a single application of the peritoneal formulation would have any effect as the active ingredient, alanyl-glutamine, would have no access to the layers of skin involved in the formation of a scar. The formula, when mixed in a regular topical cream, did not have the same effect as when mixed with an organogel such as Lipoderm™ (Transdermal vehicle, PCCA proprietary formula) or Transdel™ (Transdermal cream). Other examples of pluronic gels include but are by no means limited to Diffusimax™ and PLO™. Other suitable gels will be readily apparent to one of skill in the art.

According to an aspect of the invention, there is provided a method of treating or preventing or prophylactically treating or reducing the severity of a keloid scar comprising topically administering to a site of keloid scar formation or potential keloid scar formation an effective amount of a glutamine source. It is of note that ‘reducing the severity’ of a scar refers to ‘flattening’ or otherwise improving the appearance of a scar compared to an untreated scar of similar age and severity, as discussed herein.

In a preferred embodiment, there is provided a method of treating or preventing or prophylactically treating or reducing the severity of a keloid scar comprising topically administering to a site of keloid scar formation or potential keloid scar formation an effective amount of L-alanyl-L-glutamine in an organogel.

It is noted that ‘prophylactic treatment’ in regards keloid scar formation refers to the administration of the composition to a site at which an injury has recently occurred which is suspected of or at risk of leading to the formation of a keloid scar. Alternatively, the composition may be applied at a point at which keloid scar formation may have started at the molecular level but has not yet produced a visible scar or any visible signs of impending scar formation.

In yet another aspect of the invention, there is provided a pharmaceutical composition comprising a source of glutamine formulated or arranged for topical administration.

In a preferred embodiment, the pharmaceutical composition comprises L-alanyl-L-glutamine in an organogel.

In a preferred embodiment, the formula is as follows:
Dimethyl Sulfone 5 g
Tranilast 0.42 g
Bromelain 2 g
L-alanyl-L-glutamine 10 g
Versabase Cream qs 100 g

As will be appreciated by one of skill in the art, this formula will produce a 10% AG composition. In other embodiments, the amount of AG added may be varied, for producing a composition which is for example 5-35%, 5-30%, 10-35%, 10-30%, 5-25%, 10-25% or 20-25% AG, as discussed herein.

The invention will now be described by way of examples; however, the invention is not necessarily limited by the examples.

### EXAMPLE 1

An individual was seriously burned while having her eyebrows waxed. The eyelids obtained a first degree burn. A cream prepared as described above was applied. The cream was applied two to three times daily for three days. Peeling was expected due to the degree of burn, redness, swelling and pain to the touch however peeling did not occur. A few days after applying the cream, there was no sign of the burn.

### EXAMPLE 2

An individual with a history of keloid scars applied a topical composition as described above to a skin region soon after injury. The gash was on the forearm and was approximately 1 cm in length and several millimetres deep. Despite the fact that this individual had a history of keloid scarring, surprisingly, scar formation in the injured area was reduced compared to the expected result based on prior patient history. Specifically, while there was some discoloration, likely because the individual was in the sun when the injury occurred, the scar was flat, contrary to what was expected.

While the preferred embodiments of the invention have been described above, it will be recognized and understood that various modifications may be made therein, and the appended claims are intended to cover all such modifications which may fall within the spirit and scope of the invention.

1-5. (canceled)

6. A method of treating or preventing a keloid scar comprising topically administering to the keloid scar or a site of potential keloid scar formation an effective amount of a glutamine source.

7. The method of claim 6, wherein the glutamine source is a dipeptide comprising L-alanyl-L-glutamine.

8. The method of claim 7, wherein the dipeptide is suspended in an organogel.

9. A pharmaceutical composition for topical administration comprising glutamine and one or more pharmaceutically acceptable carriers.

10. The composition according to claim 9, wherein the glutamine is a dipeptide comprising L-alanyl-L-glutamine.

11. The composition according to claim 10, wherein the dipeptide is from about 5% to about 25% w/w of the composition.

12. The composition according to claim 11 wherein the pharmaceutically acceptable carrier comprises an organogel.
